(19) United States
(12) Patent Application Publication
(10) Pub. No.: US 2010/0306013 A1
(43) Pub. Date: Dec. 2, 2010

(54) SYSTEMS AND METHODS FOR SCHEDULING A MEDICAL SERVICE

(75) Inventors: Terilyn Mark, Layton, UT (US); Neera Gupta, Washington, DC (US); Denee Wolf, Stevensville, MD (US); Dominic Foscato, Burbank, CA (US); Tim Dobkins, Redmond, OR (US); Shay Lange, Farmington, UT (US); Amy Thomas, Logan, UT (US); Raelene Worsham, Ogden, UT (US); Sandra Shea, Clearfield, UT (US)

Correspondence Address: AUSTIN RAPP & HARDMAN, 170 South Main Street, Suite 735, SALT LAKE CITY, UT, 84101, US

(73) Assignee: IHC Health Services, Inc., Salt Lake City, UT (US)

(21) Appl. No.: 12/790,363

(22) Filed: May 28, 2010

Related U.S. Application Data
(60) Provisional application No. 61/182,236, filed on May 29, 2009.

(51) Int. Cl. G06Q 10/00

(52) U.S. Cl. 705/8

(57) ABSTRACT

A method for scheduling a medical service employing at least one computer system is described. A request for a medical service is received at the computer system. The medical service is scheduled for a requested date. At least one nonpayment exposure factor is analyzed to determine whether providing the medical service involves a nonpayment exposure to a medical service provider. The medical service may then be rescheduled for a date after the requested date if the analysis indicates nonpayment exposure to the medical service provider.

### RELATED APPLICATIONS

This application claims the benefit of U.S. Provisional Patent Application No. 61/182,236 filed on May 29, 2009. This provisional patent application is expressly incorporated herein by reference.

### TECHNICAL FIELD

The present invention relates generally to systems and methods in the medical services industry. More specifically, the present invention relates to systems and methods for scheduling a medical service.

### BACKGROUND

Currently in the medical services industry, there is a lack of procedures that are effective in obtaining the documentation necessary for effective billing and accounting procedures, particularly from those patients in need of financial assistance or charity. As a result, many medical institutions and physicians may not be fully or partially compensated for their services, or may be hindered in their ability to claim tax deductions for their expenses. These institutions and physicians may face uncompensated patient care that is increasing to an unsustainable level. Uncompensated care may include “charity care” (i.e., services provided to qualifying patients who are “unable” to pay) and “bad debt” (i.e., debt generated from providing medical services to patients who are “unwilling” to pay). These institutions and physicians may also lack the documentation necessary to access charity funds to offset the costs for their services. These institutions may also need such documentation for maintaining tax-exempt or not-for-profit status. Often, patients will only submit part of the information necessary for effective billing or tax deductions. Some difficulties include patients who refuse to complete necessary paperwork for charity care when they may otherwise qualify, patients who don't understand how to complete the form or provide any additional documentation, patients who don't know where to return the form, patients who have insurance but have a large level of personal responsibility (e.g., co-pays, etc.), undocumented patients who fear completing paperwork or patients who are noncompliant/non-residents with chronic illnesses. Furthermore, many employers may recognize that an increasing portion of their healthcare costs fund healthcare for patients who are capable of paying and therefore these employers are unhappy with medical institution collection policies. Hence, in the medical services industry, a need exists for systems and methods for scheduling a medical service based, at least partially, on nonpayment exposure of a medical service provider.

### BRIEF DESCRIPTION OF THE DRAWINGS

Exemplary configurations of the invention will become more fully apparent from the following description and appended claims, taken in conjunction with the accompanying drawings. Understanding that these drawings depict only exemplary embodiments and are, therefore, not to be considered limiting of the invention's scope, the exemplary configurations of the invention will be described with additional specificity and detail through use of the accompanying drawings in which:

FIG. 1 is a block diagram illustrating an exemplary configuration of a relationship between a patient, a physician's office, and a medical institution;

FIG. 2A is a flowchart illustrating one embodiment of a method for scheduling a medical service;

FIG. 2B is a flowchart illustrating one embodiment of a method for scheduling a medical service;

FIG. 3 is a flowchart illustrating one embodiment of a method for scheduling a medical service after an account has been established;

FIG. 4 is a block diagram illustrating one embodiment of a computer system including instructions stored in memory for scheduling a medical service;

FIG. 5A is a flowchart illustrating another embodiment of a method for scheduling a medical service;

FIG. 5B is a flowchart illustrating another embodiment of a method for scheduling a medical service;

FIG. 6 is a block diagram illustrating one embodiment of a computer system that may be employed for scheduling a medical service;

FIG. 7 is a block diagram illustrating one embodiment of a computer system, including one or more servers and computing devices, that may be utilized for scheduling a medical service; and

FIG. 8 is a block diagram illustrating one embodiment of a computer system at a physician's office and a computer system at a medical service provider for scheduling a medical service.

### DETAILED DESCRIPTION

Various configurations of the invention are described with reference to the Figures, where like reference numbers indicate identical or functionally similar elements. The configurations of the present invention, as generally described and illustrated in the Figures herein, could be arranged and designed in a wide variety of different configurations. Thus, the following more detailed description of exemplary configurations of the present invention, as represented in the Figures, is not intended to limit the scope of the invention, as claimed, but is merely representative of the configurations of the invention.

The word “exemplary” is used exclusively herein to mean “serving as an example, instance, or illustration.” Any configuration described herein as “exemplary” is not necessarily to be construed as preferred or advantageous over other configurations.

Where the described functionality is implemented as computer software, such software may include any type of computer instruction or computer-executable code located within a memory device and/or transmitted as electronic signals over a system bus or network. Software that implements the functionality associated with components described herein may comprise a single instruction, or many instructions, and may be distributed over several different code segments, among different programs, and across several memory devices.

As used herein, the terms “a configuration,” “configuration,” “configurations,” “the configuration,” “the configurations,” “one or more configurations,” “some configurations,” “certain configurations,” “one configuration,” “another configuration” and the like mean “one or more (but not necessarily all) configurations of the disclosed invention(s),” unless expressly specified otherwise.

The term “determining” (and grammatical variants thereof) is used in a broad sense. The term “determining” encompasses a wide variety of actions and therefore “determining” can include calculating, computing, processing, deriving, investigating, looking up (e.g., looking up in a table, a database or another data structure), ascertaining and the like. Also, “determining” can include receiving (e.g., receiving information), accessing (e.g., accessing data in a memory) and the like. Also, “determining” can include resolving, selecting, choosing, establishing, and the like.

The phrase “based on” does not mean “based only on,” unless expressly specified otherwise. In other words, the phrase “based on” describes both “based only on” and “based at least on.”

Systems and methods for scheduling a medical service while minimizing nonpayment exposure to a medical service provider are described in the following Figures and description. In these systems and methods, patients may pre-register for medical services. These systems and methods may be used to address the current need for improved procedures in obtaining complete and accurate patient information necessary for effective billing and accounting procedures. They may also allow a medical institution to obtain the information needed to collect on insured patient liabilities and to establish payment arrangements prior to providing medical services. They may also allow for improved medical service scheduling and attendance, patient education, verification of benefits and eligibility, and research of a patient's financial history. The systems herein may provide for the integration of clinical and financial processes and may also enable improved timing and collection amounts. Another benefit may be a reduction in the amount of bad debt as a percentage of business, and improved compliance and patient safety (e.g., identification procedures), while not compromising clinical care. The systems and methods disclosed herein may also help to ensure that patients who are capable of paying do so.

A method for scheduling a medical service employing at least one computer system is disclosed. The method includes receiving a request for a medical service at the computer system and scheduling the medical service for a requested date. The method also includes analyzing at least one nonpayment exposure factor to determine whether providing the medical service involves a nonpayment exposure to a medical service provider and rescheduling the medical service for a date after the requested date if the analysis indicates the nonpayment exposure to the medical service provider.

The scheduled date for the medical service may be maintained if the analysis does not indicate the nonpayment exposure to the medical service provider. Rescheduling the medical service for a date after the requested date may involve scheduling the medical service at least one week after the requested date.

The method may also include determining whether the medical service is urgent and if the medical service is urgent, scheduling the medical service for the requested date. The request for the medical service may be received from a second computer system. If the medical service is not urgent, the service may be provided even if the analysis indicates the nonpayment exposure to the medical service provider.

The analysis of the at least one nonpayment exposure factor may be performed automatically. The analysis of the at least one nonpayment exposure factor may include obtaining a weighted score that indicates the nonpayment exposure and comparing this score against a threshold.

A computer system for scheduling a medical service is also disclosed. The computer system includes a processor and instructions stored in memory. The instructions are executable to receive a request for a medical service at the computer system and schedule the medical service for a requested date. The instructions are also executable to analyze at least one nonpayment exposure factor to determine whether providing the medical service involves a nonpayment exposure to a medical service provider and reschedule the medical service for a date after the requested date if the analysis indicates the nonpayment exposure to the medical service provider.

A computer-readable medium including instructions stored in memory and executable by a processor for scheduling a medical service is disclosed. The instructions are executable to receive a request for a medical service at the computer system and schedule the medical service for a requested date. The instructions are also executable to analyze at least one nonpayment exposure factor to determine whether providing the medical service involves a nonpayment exposure to a medical service provider and reschedule the medical service for a date after the requested date if the analysis indicates the nonpayment exposure to the medical service provider.

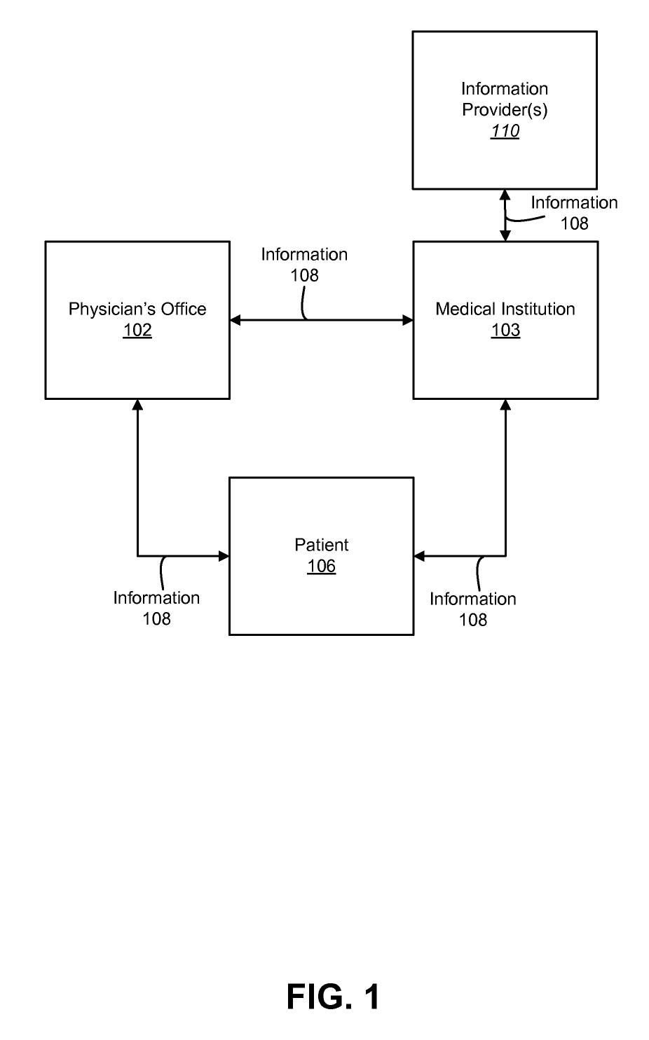

FIG. 1 is a block diagram that illustrates an exemplary configuration of a relationship between a physician's office 102, a medical institution 103, and a patient 106. The patient 106 may seek a physician for treatment at the physician's office 102. The patient 106 may communicate certain information 108 to the physician's office 102 to obtain treatment. This information may include, for example, some or all of: name, date of birth, phone number, age, sex, treatment needed/requested, symptoms experienced, primary insurance name, primary insurance policy number, etc. This information 108 may be communicated manually or in an automated fashion. For example, a patient 106 may fill out a form at the physician's office 102. A patient 106 may submit information 108 to the physician's office 102 via telephone, fax, or a computer network (e.g., Internet or intranet), etc. Additionally, the information 108 may be submitted to the physician's office 102 via computer software and optionally stored in a database.

A physician may determine that medical services from a medical institution 103 (e.g., a hospital or medical clinic) are needed or desirable. Hence, the physician's office 102 may communicate a portion or all of the patient information 108 that it has received to the medical institution 103 in order to schedule medical services for the patient 106. Generally, the medical institution 103 may defer non-urgent treatment for patients who are unwilling to provide necessary information for financial assistance and patient safety.

The medical institution 103 may require at least a certain amount of information, which may be referred to as the Minimum Information Required (MIR), before medical services are scheduled or performed. The MIR may comprise a Scheduling Data Set (SDS) and/or a Procedural Data Set (PDS). The medical institution 103 may require a SDS before medical services are scheduled. The medical institution 103 may also or alternatively require a PDS before medical services are performed. The medical institution 103 may also initiate communication with the patient 106 by phone in order to obtain additional patient information 108. This communication may occur in various ways. For example, a patient 106 may fill out a form at the medical institution 103. A patient 106 or physician may submit information 108 to the medical institution 103 via telephone, fax, or a computer network (e.g., Internet or intranet), etc. At the medical institution 103, this information may then be input into a computer system. The information 108 may also be submitted to the medical institution 103 via computer software and optionally stored in a database. In other embodiments, the patient may have previously provided the information, and as such, the information may simply be obtained from a database. In one configuration, this information 108 may be automatically obtained by computer systems communicating with the database across a computer network, such as the Internet.

As shown in FIG. 1, the medical institution 103 may also receive information from one or more information providers 110. These information providers 110 may be accessed by the medical institution 103 through the Internet or another computer network. These information providers 110 may, in some embodiments, be accessed automatically by the medical institution 103 without any user input. These information providers 110 could provide information 108 (such as financial information) regarding the patient. For example, the information providers 110 could provide information 108 to the medical institution 103 regarding the patient's credit score, credit history, payment history, any outstanding debts or loans that the patient may have, information regarding the patient's assets, bank accounts, finances, credit cards, past bankruptcies, social security information, or other types of personal information. As will be explained herein, this financial or other information 108 may be used by the medical institution 103 in assessing the risk whether or not the patient is likely to pay (or timely pay) for requested medical services.

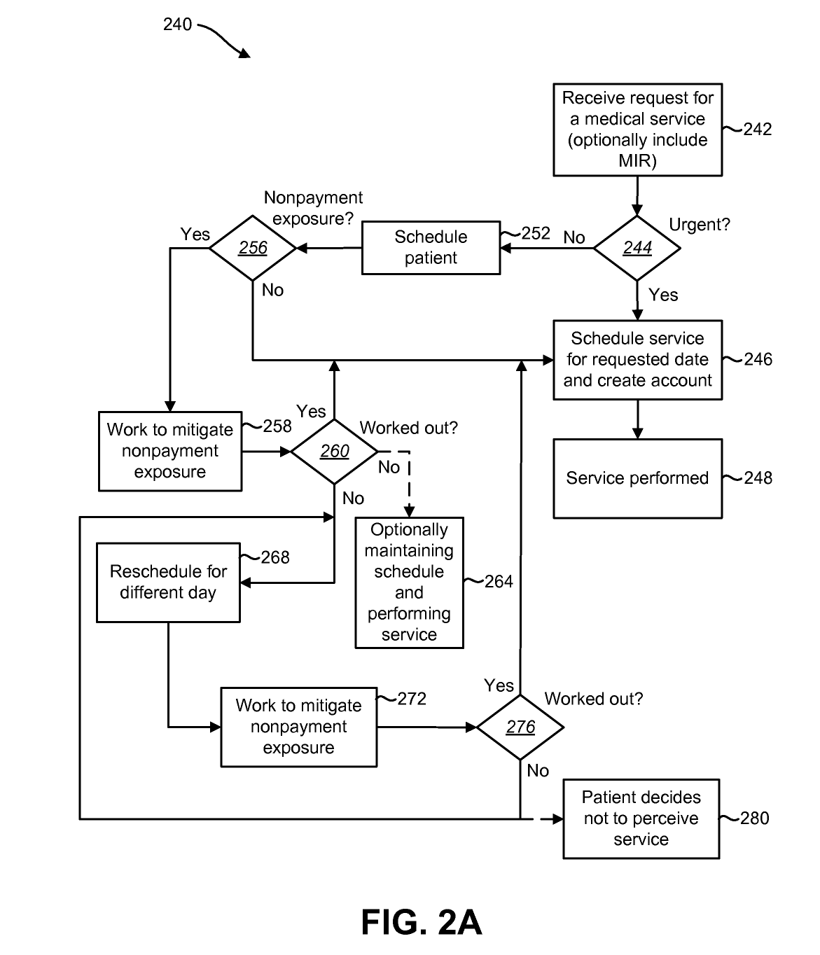

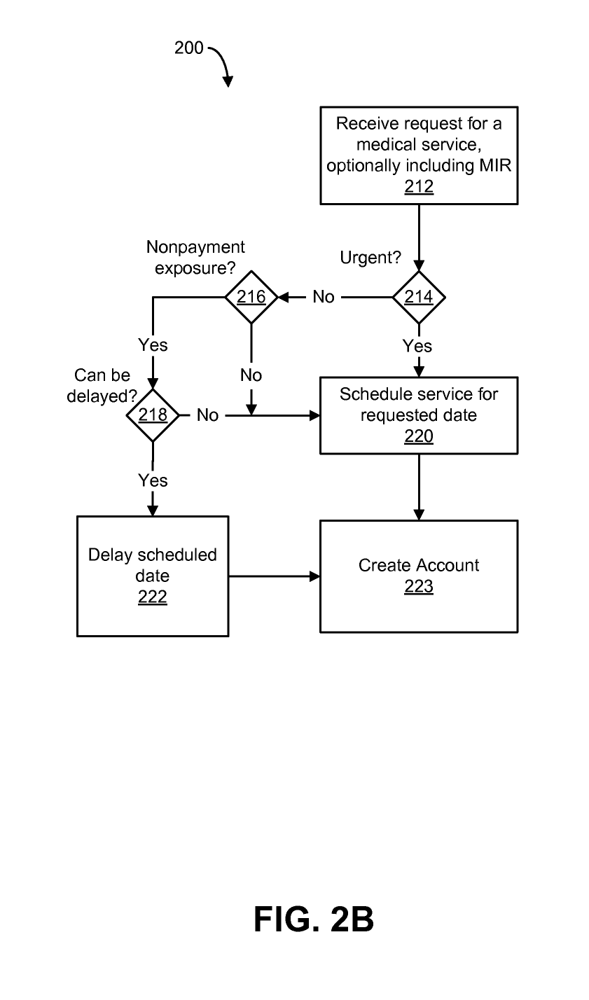

FIG. 2A is a flowchart that illustrates an exemplary configuration of a method 240 for scheduling desired medical services. A physician's office 102 may collect some or all of the information 108 or items on the MIR from a patient 106. An MIR may include certain pieces of patient information 108, e.g., name, date of birth, social security number, home phone number, address, insurance name, insurance billing information, insurance card information, physician name, etc. An MIR may also include additional information, e.g., admitting diagnosis, reason for procedure, medical procedure, procedure code, pre-authorization/authorization number, desired schedule for medical service, whether or not the service may be deferred, etc. The physician's office 102 may receive this information 108 in various ways (e.g., questioning the patient directly, or having the patient submit electronic information via the Internet, etc.).

The medical institution 103 may then receive 242 a request for a medical service from the physician. The medical institution 103 may require all or part of the MIR to schedule the medical service. The request and MIR may be received 242 simultaneously or at separate times by the medical institution 103. The request and MIR may be received 242 in various ways (e.g., the request may be input in a computer system based on information 108 received from the physician's office 102 or patient 106 via telephone, fax, computer network (e.g., Internet, intranet, etc.), mail, or by any other method) or may be input into an electronic form by the patient 106 or medical personnel. In some embodiments, a patient 106 may be precluded from obtaining (or scheduling) medical service without the MIR. At this step (242), a physician may indicate the urgency of service and may recommend that the requested medical service not be rescheduled.

It may then be determined 244 whether the medical service is urgent, based on, for example, information 108 from the physician. The physician may determine 244 whether the case is urgent depending on medical necessity, an amount of time within which treatment is necessary or desired, or other factors. Whether the physician indicates that the case is urgent may preclude a reschedule or delay of service later in the process. If the physician indicates that the case is urgent, then service may be scheduled 246 for the date requested and an account may be created. The service may then be performed 248 on the patient 106 (e.g., the patient receives the requested medical service). If it is indicated that the case is urgent, medical institution personnel may input notes on the case within a computer system, or notes may be generated automatically via computer software and/or saved in a database. Information 108 received from a physician may be a primary factor in determining whether the patient ultimately receives medical services.

If it is determined 244 that the case is not urgent, then the patient 106 may be scheduled 252 for receiving the desired medical service on a requested (specified) date. Once the medical service has been scheduled 252 with the medical institution 103, the particular case may be screened (analyzed) to determine 256 whether providing the service involves a nonpayment exposure to the medical institution 103. As used herein, the term “nonpayment exposure” refers to financial exposure or risk due to nonpayment or reduced payment by a patient or insurance company for a medical service. The factors that may be used to determine the nonpayment exposure are described herein. It should be noted that the process of analyzing 256 loss exposure may be done in an automated fashion, e.g., automatically by the medical institution 103 (or more specifically by a computer owned by the medical institution 103) without any user input or user authorization. In conducting this analysis of whether there is nonpayment exposure, information 108 obtained from the providers 110 may be used. In fact, as part of this process, the information 108 (such as financial or credit information from online credit bureaus) may be obtained automatically by one or more computer systems via a computer network as part of the nonpayment exposure analysis.

If the screening 256 indicates that the case does not include a nonpayment exposure, then service may be scheduled 246 for the date requested and an account may be created.

If the screening 256 indicates that the case includes a nonpayment exposure, then the medical institution 103 may work 258 to mitigate the nonpayment exposure. As described herein, this work 258 may involve working with the patient 106 or the physician to provide additional information 108 (such as information necessary to obtain charity or Medicaid or other insurance coverage for the service), obtaining authorization for the procedure from the insurance company, getting the patient 106 to resolve previous debt to the medical institution 103 or to a physician, having the patient pre-pay all or a portion of the bill, etc.

If by working 258 with the patient/physician the nonpayment exposure is mitigated 260, the service may be scheduled 246 for the date requested and an account may be created and the service may be performed 248. If the patient is unable to work out (mitigate) 260 the nonpayment exposure, the medical institution 103 has the option of maintaining 264 the date previously scheduled for the service (or perhaps rescheduling) and ultimately performing the service. However, in an alternative scenario when the patient is unable to mitigate 260 the nonpayment exposure, the medical service may be rescheduled 268 for a later date (such as a date that is at least one week later than the previously-scheduled date).

Once rescheduled, the patient 106/physician can continue to work 272 to reduce the nonpayment exposure. If this nonpayment exposure is ultimately worked out 276, the service may be scheduled 246 for the date requested and an account may be created. Otherwise, the medical institution 103 can continue to reschedule 268 the date until the nonpayment exposure may be worked out or the patient decides 280 not to receive the service. Of course, at any time, the medical institution 103 could opt to take the risk and perform the service 264.

FIG. 2B is a flowchart that illustrates an exemplary configuration of a method 200 for scheduling medical services. A physician's office 102 may collect some or all of the information 108 or items on the MIR from a patient 106. An MIR may include certain pieces of patient information 108, e.g., name, date of birth, social security number, home phone number, address, insurance name, insurance billing information, insurance card information, physician name, etc. An MIR may also include additional information, e.g., admitting diagnosis, reason for procedure, medical procedure, procedure code, pre-authorization/authorization number, desired schedule for medical service, whether or not the service may be deferred, etc. The physician's office 102 may receive this information 108 in various ways (e.g., questioning the patient directly, or having the patient submit electronic information via the Internet, etc.).

The medical institution 103 may then receive 212 a request for a medical service from the physician. The medical institution 103 may require all or part of the MIR to schedule the medical service. The request and MIR may be received 212 simultaneously or at separate times by the medical institution 103. The request and MIR may be received 212 in various ways (e.g., the request may be input in a computer system based on information 108 received from the physician's office 102 or patient 106 via telephone, fax, computer network (e.g., Internet, intranet, etc.), mail, or by any other method) or may be input into an electronic form by the patient 106 (or the patient's parent(s), associated, etc.) or medical personnel. A patient 106 may be precluded from obtaining medical service without the MIR. At this step (212), a physician may indicate the urgency of service and may recommend an exception to rescheduling a patient for treatment, as necessary. It should be noted that the physician may, at any time, designate a service as being urgent. For example, after a date for a service has been rescheduled (as described herein) the physician may step in and order the service and/or declare that the service is urgent. The physician may deem the service urgent at any other time during the treatment of the patient.

It may be then be determined 214 whether the medical service is urgent, based on, for example, information 108 from the physician. The physician may determine 214 whether the case is urgent depending on medical necessity, an amount of time within which treatment is necessary or desired, or other factors. Whether the physician indicates that the case is urgent may preclude a reschedule or delay of service later in the process. If the physician indicates that the case is urgent, then service may be scheduled 220 for the date requested and an account may be created 223. If it is indicated that the case is urgent, medical institution personnel may input notes on the case within a computer system, or notes may be generated automatically via computer software and/or saved in a database. Information 108 received from a physician may be a primary factor in determining whether the patient ultimately receives medical services.

If it is determined 214 that the case is not urgent, then the case may be screened to determine 216 whether providing the service involves a nonpayment exposure. (In some embodiment, a date may be scheduled and then the analysis performed). A subset of loss exposure factors may include whether the treating facility/physician is “Out of Network” (e.g., a particular medical institution 103 is considered outside of the patient's insurance network), whether authorization is needed for treatment (e.g., the patient 106 needs authorization from his/her insurance company and/or physician for treatment), whether authorization is not obtained within a predetermined period before scheduled treatment, and/or whether the case is “Self Pay” (e.g., the patient intends to pay the medical bill directly). The subset of loss exposure factors may be obtained from or determined based on the MIR at scheduling, or at any other point in the process. Other factors that may indicate a nonpayment exposure may include whether the patient 106 already has a predetermined amount of bad debt (with the medical institution 103 or otherwise), insurance with high patient liability, lack of insurance authorization, previous outstanding balances, whether Worker's Compensation/Third-Party-Liability (TPL) coverage information is missing, a patient's previous failure to pay other third parties, a patient's poor credit scores, the income level of the patient (and more specifically a low income level of the patient), or if the patient liability will be more than a predetermined amount of money. (Some of the data considered by these factors may be obtained from the providers 110 automatically by the computer systems.) In one example, the predetermined amount of patient liability to indicate nonpayment exposure may be anything in excess of $1,000, and may apply whether or not the patient is insured. The process of analyzing 216 loss exposure may be done in an automated fashion. For example, billing system information may be available during the admission process to determine whether a patient has a predetermined amount of bad debt (with the medical institution or otherwise). If the screening indicates that the case does not include a nonpayment exposure, then service may be scheduled 220 for the date requested and an account may be created 223.

If the screening indicates that the case includes a nonpayment exposure, then it may be determined 218 whether service can be scheduled at a later date. The later date may be, in one embodiment, at least one week after the requested date.

At this step (218), a physician may indicate the urgency of service and may recommend an exception to rescheduling a patient 106 for treatment as necessary. Hospital personnel may contact the physician's office 103 manually (e.g., by phone, fax, mail, in person, etc.) to schedule the service, or the service may be scheduled in an automated fashion (e.g., via computer software). If it is determined 218 that service cannot be scheduled at a later date due to urgency, then service may be scheduled 220 for the date requested and an account may be created 223. If it is determined 218 that service can be scheduled at a later date, then service may be scheduled 222 at a later date, such as at least one week after the requested date, and an account may be created 223. The service may be scheduled, for example, one week in the future. This delay may afford time to financially clear a patient 106 for medical service or take other steps, as appropriate.

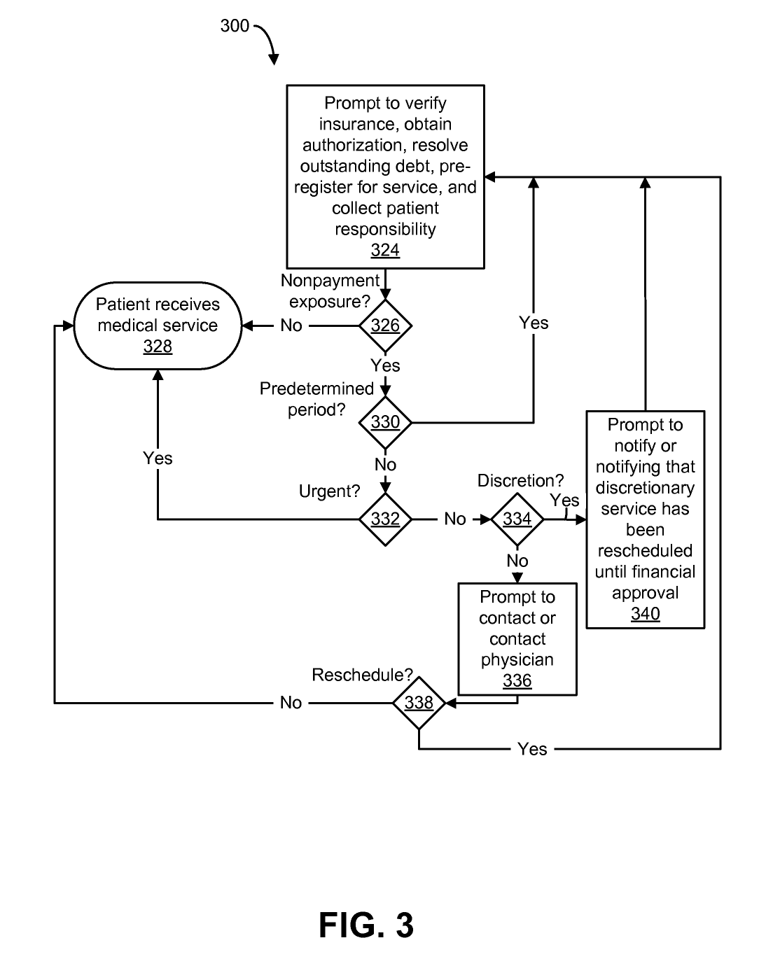

FIG. 3 is a flowchart illustrating one embodiment of a method 300 for scheduling a service after an account has been created, for example, as shown in FIGS. 2A and 2B. A computer system may prompt 324 personnel to work with the patient 106, physician's office 102, and/or the insurance company to determine any patient liability for the service, obtain authorization, research the patient's previous financial assistance information, research and resolve outstanding bad debt with the medical institution, collect patient responsibility, confirm the medical service the patient is scheduled for, confirm the scheduled date and time, confirm whether the patient qualifies for discounted/free care, confirm what services are discounted and discount levels offered, and/or confirm patient identification and appropriateness of admissions. The medical institution 103 may provide the patient 106 with a cost estimate for the medical service, and/or provide an estimate of patient responsibility based on the patient's insurance coverage. The medical institution 103 may seek an agreement with the patient 106 as to how the services will be paid and may provide financing options. The medical institution 103 may collect a deposit or payment for medical service. Such a full or partial payment may be requested from all patients, and/or of required “high-financial-risk” patients. A discount may be offered for automated payment plans. The patient 106 may choose to reschedule the medical service due to the cost of the service. The patient 106 may also choose to reschedule the service for a later time if he/she has not met his/her insurance deductible.

A computer system may prompt 324 personnel to take the identified actions in various ways, such as by displaying a message on a computer monitor or by creating a task list that may be displayed on a computer monitor or printed.

The medical institution 103 may also offer the patient 106 financial assistance or charity based on financial and demographic information 108 (e.g., patient income, family size, etc.) received from the patient 106. The medical institution 103 may designate a patient to “Self-Pay” status if the patient 106 does not submit adequate information 108 within a predetermined period. This predetermined period may be 10 days. The medical institution 103 may work with a patient 106 to apply for, determine, or acquire financial assistance (e.g., Medicaid, charity care, discounts, payment plans, etc.). Access to financial assistance may depend on income, and/or credit scoring. The medical institution 103 may discuss financial obligations with the patient 106 or the patient's family and seek an agreement on payment. The medical institution 103 may access information 108 (manually or in an automated fashion) pertaining to a patient's earlier commitment(s) to submit financial assistance information or “bad debt” history. The medical institution 103 may inform physician's offices 102, clinical departments (medical institutions), or patients 106 as to whether financial issues are resolved, and/or such information regarding resolution of the issues may be stored in a computer database and associated with the patient's records.

Uninsured patients may be required to set up or participate in an initial financial screening discussion or have their service rescheduled or deferred. An uninsured patient may have the option of paying estimated responsibility in full on the date of service to avoid deferment. An uninsured patient may agree to pay in full when receiving his/her first statement (a predetermined deposit percentage may be required, depending on patient financial history) to avoid deferment of service. An uninsured patient may set up a payment plan that may be short or long term (with predetermined terms, interest amounts, minimum payments, and initial payment requirements) to avoid deferment of medical service. Patients with high estimated patient responsibility may have options similar to uninsured patients to avoid medical service deferment. Patients with bad debt may pay an estimated responsibility in full, or submit necessary financial assistance information to avoid deferment of medical services. They may also be required to resolve all or part of their bad debt accounts or medical services may be deferred. Lack of information or agreement may preclude a patient from receiving medical service or may cause the patient's case to be delayed (e.g., rescheduled) until proper information or agreement is received. Certain categories of patients may be exempted from financial assistance policy (FAP) compliance, urgent determinations, loss exposure assessment/screening, and/or medical service deferment (e.g., children, catastrophic medical necessity, pregnant women, mental health patients, etc.).

It may then be determined 326 whether the case includes nonpayment exposure. The factors to determine 326 whether a case includes a nonpayment exposure may be the same or similar to the possible earlier screening process of FIGS. 2A and 2B (such as process 256). This process 326 may be done in an automated fashion. If it is determined 326 that the case does not include nonpayment exposure, then the patient 106 may receive 328 the medical service. If it is determined 326 that the case includes nonpayment exposure, then it may be determined 330 whether the date of scheduled service is within a predetermined period in the future. The predetermined period may be at two days, within two days, or any other period. This determination 330 may be made in an automated fashion employing a computer system. If it is determined 330 that the date of scheduled service is within a predetermined period in the future, then personnel may be prompted 324 to work with a patient 106, physician's office 102, and/or the insurance company to obtain authorization, resolve outstanding bad debt, and/or collect patient responsibility.

If it is determined 330 that the date of service is not within a predetermined period in the future, then it may be determined 332 whether the case is urgent. As noted, this determination 332 may be made in response to information 108 received from a physician. The physician may recommend an exception to rescheduling a patient 106 for treatment, as necessary. Urgent cases may be initially scheduled three days or less from the date of contact. If it is determined 332 that a case is urgent, then the patient 106 may receive 328 service. “Out of Network” patients may be referred to in-network facilities, as appropriate.

If it is determined 332 that the case is not urgent, then it may be determined 334 whether the service is discretionary. Discretionary services may include highly specialized, elective, extraordinary services (e.g., organ or bone marrow transplants), cosmetic procedures, experimental treatment, refractive eye surgery, gene therapy, hyperbaric treatment, outpatient kidney dialysis, treatment for obesity, and other procedures (e.g., plastic surgery, carpal tunnel syndrome, etc.). Discretionary services may not include basic diagnostic or therapeutic services generally performed by local providers. If it is determined 334 that the service is discretionary, then personnel may be prompted 340 (such as by a message on a computer display screen or task list) to contact a physician and notify the physician that the discretionary service has been rescheduled until it may be financially approved. The physician may also be notified in an automated fashion (e.g., by an automatically generated fax or electronic message). As such, patients 106 may be precluded from obtaining discretionary treatment until their case is financially cleared. The medical institution 103 may be prompted 324 to work with a patient 106, physician's office 102, and/or the insurance company to obtain authorization, resolve outstanding bad debt, and/or collect patient responsibility. If it is determined 334 that the service is not discretionary, then personal may be prompted 336 to contact a physician to determine 338 whether to reschedule the medical service. A physician may indicate the urgency of service and may recommend an exception to rescheduling a patient 106 for treatment as necessary. Also, the physician may be contacted in an automated fashion, such as by an automatically generated fax, telephone call, or electronic message. The rescheduled date may be, for example, one week after the rescheduled date. If it is determined 338 that the service cannot be rescheduled for a later date, then the patient may receive 328 the medical service. If it is determined 338 that the service may be rescheduled for a later date, then the medical institution may be prompted 324 to work with a patient 106, physician's office 102, and/or the insurance company to obtain authorization, resolve outstanding bad debt, and/or collect patient responsibility. When a patient 106 receives 328 medical service, the patient 106 may report to the medical institution 103 on or before their scheduled time, check in at the medical institution desk, pay a deposit (if not previously collected), and receive 328 the scheduled medical service.

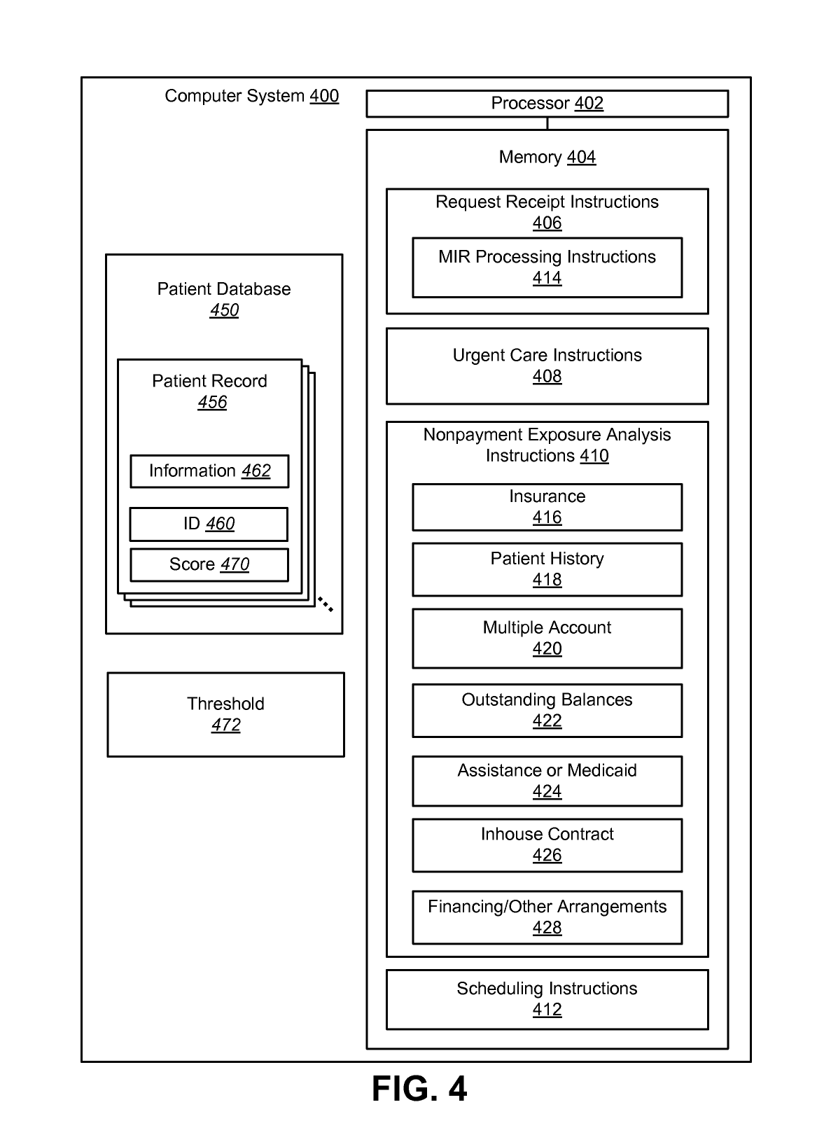

FIG. 4 is a block diagram illustrating one embodiment of a computer system 400 including instructions stored in memory for scheduling a medical service. The computer system 400 of FIG. 4 includes a processor 402 and memory 404. For simplicity, other components (such as a display screen, keyboard, and mouse) are not illustrated in FIG. 4, but could be included within or used by a computer system 400. Instructions stored in the memory 400 may be executed by the processor 402 to perform various functions, as will be explained below.

The instructions stored in memory 404 may include request receipt instructions 406, urgent care instructions 408, loss exposure analysis instructions 410, and scheduling instructions 412. The request receipt instructions 406 comprise instructions executable by the processor 402 that enable, for example, a user to input a request for a medical service remotely via a network, at the computer system 400 using, for example, a keyboard and mouse, or via by transmission from a second computer system.

The request receipt instructions 406 may further comprise MIR processing instructions 414. The MIR processing instructions 414 may enable the input of all Medically Required Information (MIR) and, in one embodiment, prevent scheduling of a medical service (through interacting with the scheduling instructions 412) if all the required medical information 108 is not received.

The urgent care instructions 408 are instructions configured to receive input or data indicating whether the pertinent medical service is urgent in nature. This input or data may be received at the computer system 400 or may be received from a remote system. The urgent care instructions 408 interact with the scheduling instructions 412, in one embodiment, to prevent or delay scheduling of a medical service if the medical service is not urgent and the loss exposure analysis instructions 410 involve a nonpayment exposure in providing the service.

The loss exposure analysis instructions 410 comprise instructions that analyze the nonpayment exposure in relation to providing the pertinent medical service. For example, the loss exposure analysis instructions 410 analyze various factors, such as those enumerated above, and, whether insurance coverage 416 is available for the medical service (including whether the “Out-of-network” coverage is sought, whether insurance authorization is needed, whether Worker's Compensation/Third-Party-Liability (TPL) coverage is available, the total patient liability, or whether the patient 106 intends to pay for the procedure without the assistance of insurance), the patient history 418 for the patient 106, whether the patient 106 at issue has multiple accounts/bad debts 420, any outstanding balances 422 for the patient 106, whether assistance or Medicaid 424 is requested for the medical service, whether an inhouse contract 426 may cover the procedure, or whether other financing or other arrangements 428 have been made for payment of the medical service. These factors 416, 418, 420, 422, 424, 426, 428 may be analyzed in various ways. For example, data may be gathered from other systems or requested to be input, or simply input, in order to analyze these factors 416, 418, 420, 422, 424, 426, 428.

The scheduling instructions 412 may interact with the request receipt instructions 406, urgent care instructions 408, and loss exposure analysis instructions 410 to determine when the medical services are or may be scheduled. For example, if all the required medical information 108 is not provided, as indicated by the MIR processing instructions 414, the medical service will not be scheduled or will be delayed, in one embodiment. Also, for example, if the urgent care instructions 408 indicate that the medical service is urgently required, the medical service may be scheduled, even if there is a nonpayment exposure as indicated by the loss exposure analysis instructions 410. Also, in one embodiment, the medical service will not be scheduled or will be delayed by the scheduling instructions 412 if there is a risk of loss exposure based on the analysis of the loss exposure analysis instructions 410 and if the urgent care instructions 408 do not indicate that urgent care is required. The scheduling instructions 412 may also interact with or include calendar data indicating the availability of medical facilities, equipment, and personnel.

It should also be noted that each of the sets of instructions 406, 408, 410, 412 are not necessarily mutually exclusive. For example, common programming code or programming algorithms may be implemented, utilized or accessed by the different instructions 406, 408, 410, 412.

FIG. 4 further indicates that there may be a database 450 that includes records 456 regarding each particular patient 106. The information 108 obtained from the patient 106 or the physician may be entered into the database 450. This database 450 may be stored on the computer system 400 or on another computer system. If the database 450 is on another system, the computer system 400 may be configured to communicate with the database 450 and/or receive information from the database 456 via a computer network, the Internet, etc.

Each record 456 in the database 450 may include a patient ID 460 as well as information 462 (in one or more fields) that may be used in analysis the nonpayment exposure. Again, this information 462 may include the patient's financial information, payment information, credit information, etc. Some of this information 462 may be obtained (or even automatically obtained) from the providers 110.

In some embodiments, the database 450 may also include a nonpayment exposure score field 470. For example, the data regarding the nonpayment risk analysis may be compiled and manipulated into a numeric value, such as a score 470, that will be used to evaluate the nonpayment risk. This may be accomplished by having the analysis instructions 410 weigh (as desired) the various factors and combine them into a numeric score 470 that may be used to rapidly judge the nonpayment exposure risk associated with a particular patient 106. This nonpayment analysis score 470 may be compared against established thresholds 472 to determine the nonpayment exposure of a particular patient 106. Additionally or alternatively, the nonpayment risk may be summed and evaluated into non-numeric ranges such as “low” nonpayment risk, “medium” nonpayment risk, “high” nonpayment risk, etc. Other ways of manipulating and/or analyzing the nonpayment risk based upon the underlying information 462 may also be used. The score 470 or rating of the patient 106 may be saved in a database 450 and/or passed onto the physician. In some embodiments, the patient's nonpayment risk score 470 may be automatically generated and compared against the thresholds 472 without any user input.

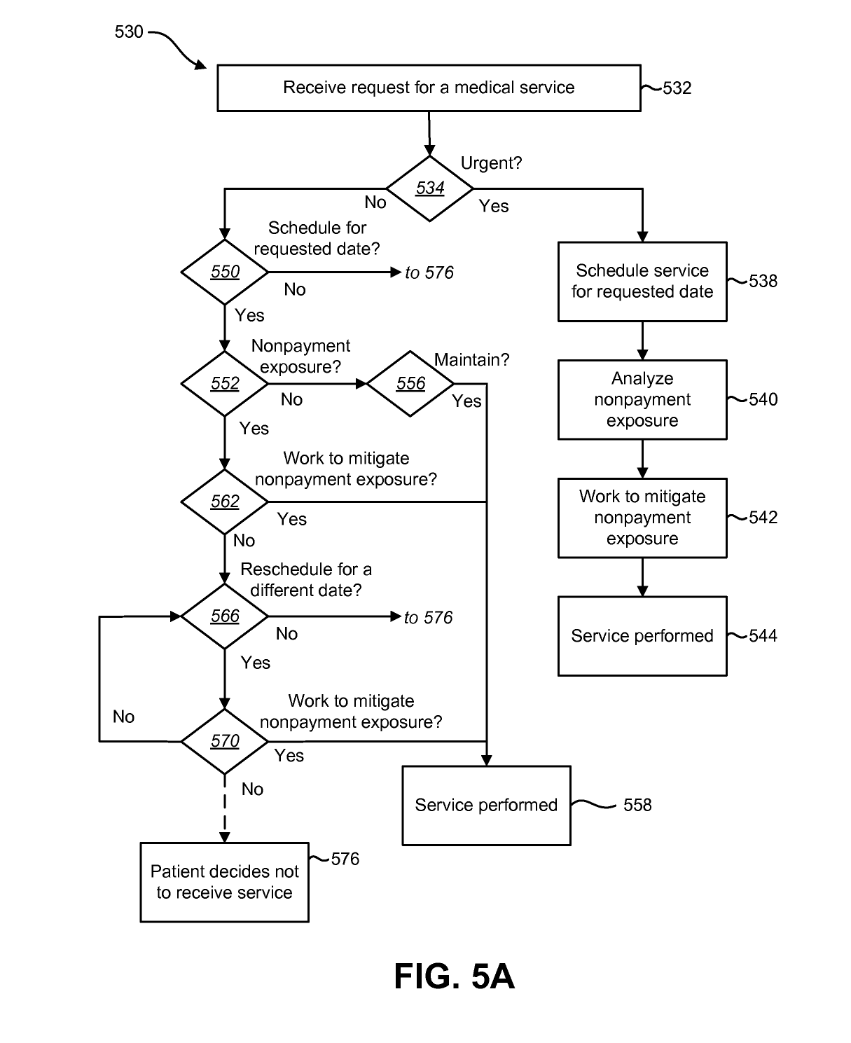

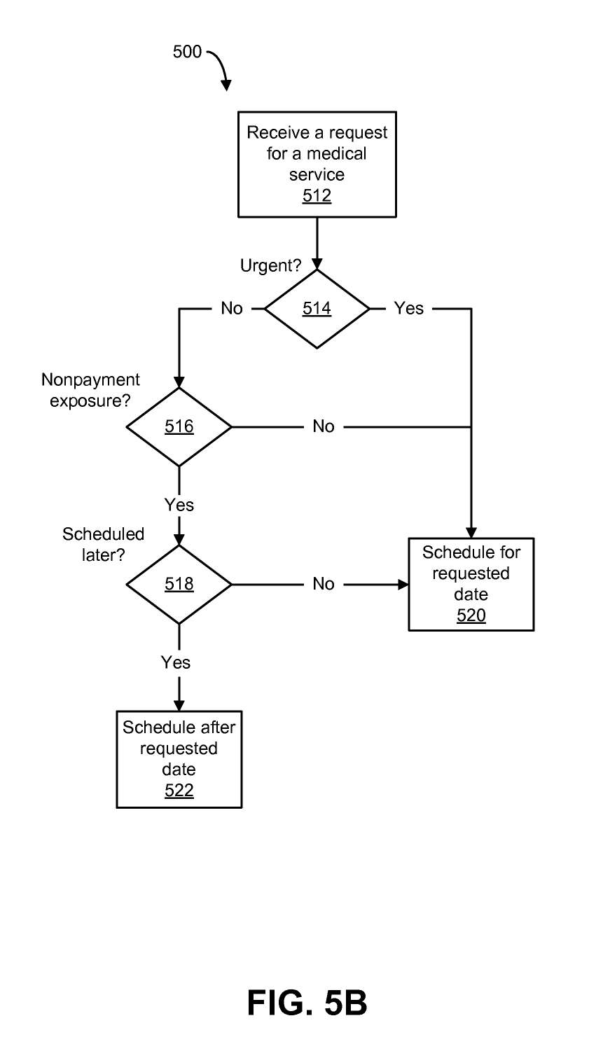

FIG. 5A is a flowchart illustrating another embodiment of a method 530 for scheduling a medical service. In the illustrated method 530, a request for medical service is received 532 at a computing device. The request may optionally include MIR information. The MIR may be received at a separate time from the request and may be received at various times. It may then be determined 534 whether the request for medical services is of an urgent nature. If the request is urgent, the medical service is scheduled 538 for the requested date. Once scheduled, a nonpayment analysis may be performed 540 to determine whether providing the medical service involves a nonpayment exposure to a medical service provider. This nonpayment analysis may be of the type described herein and may consider at least one of the factors enumerated in connection with the nonpayment exposure instructions 410 of FIG. 4. As described above, these factors may include insurance considerations 416, patient history 418, multiple account/bad debts 420, outstanding balances 422, assistance to be provided or Medicaid 424, whether the medical service is to be covered by an in-house contract 426, and financing or other arrangements for payment 428. If it turns out that there is a risk of nonpayment exposure, the medical institution 103 may work 542 with the patient 106 or physician to mitigate this exposure (as described herein). However, because the service is urgent, the service may be performed 544, even if the nonpayment exposure cannot be mitigated/worked out 542.

If the request for the medical service is not urgent in nature, the service may be scheduled 550 for a requested date. If it is determined 550 not to schedule for a requested date (e.g., a patient declines to schedule), the patient may not receive service 576. However, if it is determined 550 to schedule for a requested date, a nonpayment exposure analysis may be performed 552. If this analysis indicates that there is no nonpayment exposure, the scheduled date for the service may be maintained 556 and the service may be performed 558.

If the nonpayment analysis indicates that there is a nonpayment risk, then the medical institution 103 may work 562 with the patient 106 or physician, as described herein, to mitigate this exposure. If working 562 with the patient 106 results in the risk being mitigated, the service may be performed 558. If working 562 with the patient 106 does not result in the nonpayment risk being mitigated, the medical service may be rescheduled 566 for a later date, such as, for example, a date that is at least one week later than the previously-scheduled date. If it is determined 566 not to reschedule (e.g., the patient declines to reschedule), the patient may not receive service 576. In some embodiments, the date of the service will automatically be rescheduled 566 if it is determined that the patient owes the medical institution money, has bad debt to the institution, or has failed to pay the institution in the past. After the rescheduling 566 of the scheduled date, the medical institution may continue to work 570 to mitigate the risk, with the outcome hopefully being that the risk is mitigated and the service is performed 558. As shown by FIG. 5A, the date may be rescheduled 570 as many times as necessary for the risk to be mitigated. However, if the risk cannot be mitigated, the patient may ultimately decide 576 not to receive the service.

FIG. 5B is a flowchart illustrating another embodiment of a method 500 for scheduling a medical service. In the illustrated method 500, a request for medical service is received 512 at a computing device. The request may optionally include MIR information. The MIR may be received at a separate time from the request and may be received at various times. It is then determined 514 whether the request for medical services is of an urgent nature. If the request is urgent, the medical service is scheduled 520 for the requested date.

If the request for the medical service is not urgent in nature, at least one nonpayment exposure factor is analyzed 516 to determine whether providing the medical service involves a nonpayment exposure to a medical service provider. This analysis may consider for example, the factors enumerated in connection with the nonpayment exposure instructions 410 of FIG. 4, such as insurance considerations 416, patient history 418, multiple account/bad debts 420, outstanding balances 422, assistance to be provided or Medicaid 424, whether the medical service is to be covered by an inhouse contract 426, and financing or other arrangements for payment 428. If it determined 516 that providing the service does not involve a financial exposure loss, the medical service is scheduled 520 for the requested date.

If it is determined 516 that providing the medical service involves a nonpayment exposure, it is determined 518 whether the medical service may be scheduled later. If it cannot be scheduled later, the medical service is scheduled 520 for the requested date. If it may be scheduled later, the medical service is scheduled 522 after the requested date, for example, one week following the requested date.

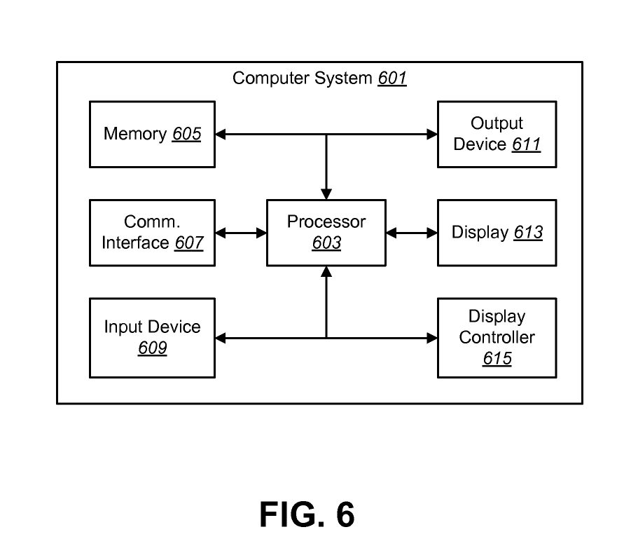

FIG. 6 is a block diagram illustrating one embodiment of a computer system 601 that may be employed for scheduling a medical service. The illustrated components may be located within the same physical structure or in separate housings or structures.

The computer system 601 includes at least one processor 603 and memory 605. The processor 603 controls the operation of the computer system 601 and may be embodied as a microprocessor, a microcontroller, a digital signal processor (DSP) or other device known in the art. The processor 603 typically performs logical and arithmetic operations based on program instructions stored within the memory 605.

As used herein, the term memory 605 is broadly defined as any electronic component capable of storing electronic information, and may be embodied as read-only memory (ROM), random access memory (RAM), magnetic disk storage media, optical storage media, flash memory devices in RAM, on-board memory included with the processor 603, EPROM memory, EEPROM memory, registers, etc. The memory 605 typically stores program instructions and other types of data. The program instructions may be executed by the processor 603 to implement some or all of the methods or functions disclosed herein.

The computer system 601 typically also includes one or more communication interfaces 607 for communicating with other electronic devices. The communication interfaces 607 may be based on wired communication technology, wireless communication technology, or both. Examples of different types of communication interfaces 607 include a serial port, a network adaptor, a parallel port, a Universal Serial Bus (USB), an Ethernet adapter, an IEEE 1394 bus interface, a small computer system interface (SCSI) bus interface, an infrared (IR) communication port, a Bluetooth wireless communication adapter, and so forth.

The computer system 601 typically also includes one or more input devices 609 and one or more output devices 611. Examples of different kinds of input devices 609 include a keyboard, mouse, microphone, remote control device, button, joystick, trackball, touchpad, lightpen, etc. Examples of different kinds of output devices 611 include a speaker, printer, etc. One specific type of output device which is typically included in a computer system is a display device 613. Display devices 613 used with embodiments disclosed herein may utilize any suitable image projection technology, such as a cathode ray tube (CRT), liquid crystal display (LCD), light-emitting diode (LED), gas plasma, electroluminescence, or the like. A display controller 615 may also be provided, for converting data stored in the memory 605 into text, graphics, and/or moving images to be shown on the display device 613.

Of course, FIG. 6 illustrates only one possible configuration of a computer system 601. Various other architectures and components may be utilized.

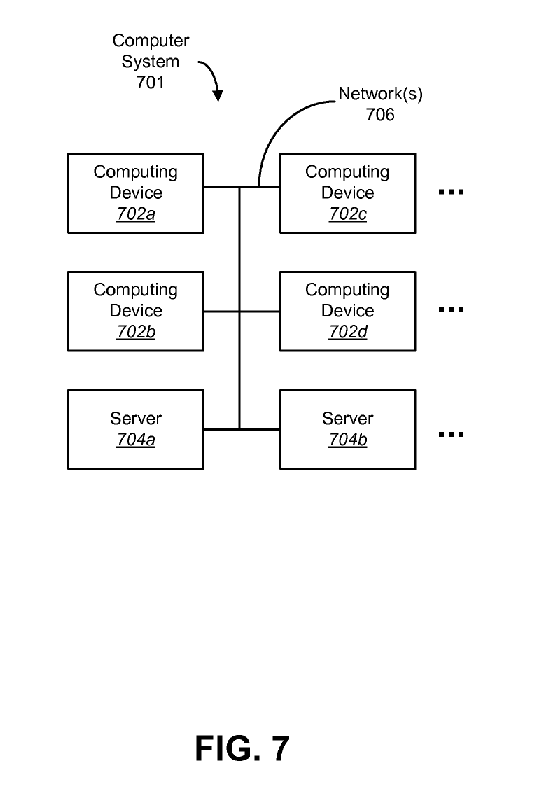

FIG. 7 is a block diagram illustrating one embodiment of a computer system 701, including various computing devices 702a-d and servers 704a-b, that may be utilized for scheduling a medical service. The computing devices 702a-d and servers 704a-b may be located at the same or different geographical locations, such as at different office buildings, in different cities, or in different states or countries. The computing devices 702a-d and servers 704a-b may be in communication via one or more networks 706, which may include the Internet. As a result, request for a medical service may be input from various locations on the computer system 701 and processed at different locations utilizing different computing devices 702a-d and/or servers 704a-b.

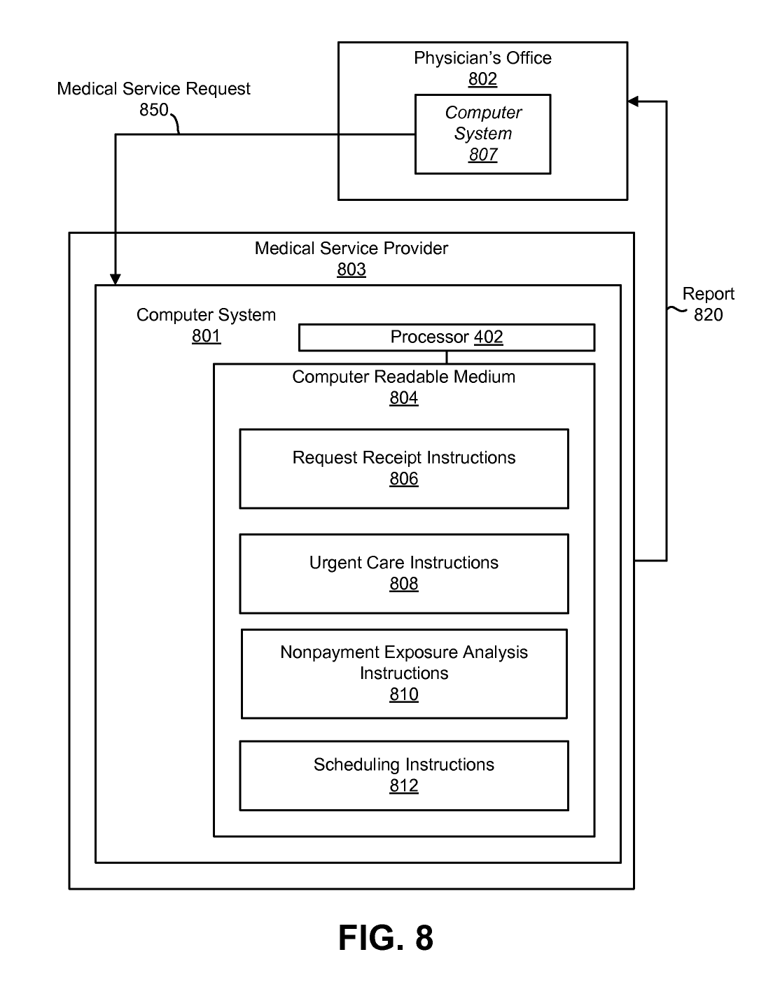

FIG. 8 is a block diagram illustrating one embodiment of a computer system 807 at a physician's office 802 and a computer system 801 at a medical service provider 803 (e.g., a hospital) for scheduling a medical service. More specifically, FIG. 8 illustrates a computer system 807 at the physician's office 802 for transmitting the medical service request 850 to a medical service provider 803. As illustrated in FIG. 8, the medical service request 850 is transmitted to the computer system 801 of the medical service provider 803. Also, as explained in greater detail in connection with FIG. 4, the processor 402 may process instructions stored on a computer readable medium 804 (e.g., memory), such as request receipt instructions 806, urgent care instructions 808, financial risk analysis instructions 810, and scheduling instructions 812 to schedule a medical service and manage information.

As shown by arrow 820, a report can be sent from the provider 803 to the physician's office 802 regarding the nonpayment exposure created by this case. This report 820 may be sent automatically by the computer system 801 and may be received by the computer system 807. This report 820 may include all of the factors, results, information used by the processor 402 in constructing the nonpayment exposure analysis. This report 820 may include any and/or all of the specific data used in the analysis and even data (such as financial data or credit scores) obtained from the third party providers 110. Alternatively, the report 820 may simply include information regarding the nonpayment exposure, the determined composite nonpayment score of the patient, the threshold value, or an indication regarding the level of nonpayment exposure risk created by the patient (e.g., low, medium, high, etc.). The reasons why a patient 106 creates a particular nonpayment risk may also be provided, as desired.

The illustrated embodiment depicts only one configuration of the disclosed systems and methods. For example, a medical service request 850 could be transmitted to multiple medical service provider 803 by electronic means. Alternatively, a medical service request 850 could be transmitted via facsimile to one or more medical service providers 803. Thereafter, at the medical service provider 803, the medical information request, and optionally MIR, may be input into a computer system 801 by, for example, a person.

The systems and methods described herein enable medical institutions and personnel to more effectively obtain and verify the documentation necessary to mitigate the nonpayment exposure associated with patient liability. They allow medical institutions and personnel to better document financial assistance or charity cases, such that expenses may be claimed as tax deductions or offset with charity funds. They also allow medical institutions the ability to obtain documentation necessary to prove not-for-profit status.

The various illustrative logical blocks, modules, and circuits described in connection with the configurations disclosed herein may be implemented or performed with a general purpose processor, a digital signal processor (DSP), an application specific integrated circuit (ASIC), a field programmable gate array signal (FPGA) or other programmable logic device, discrete gate or transistor logic, discrete hardware components, or any combination thereof designed to perform the functions described herein. A general purpose processor may be a microprocessor, but in the alternative, the processor may be any conventional processor, controller, microcontroller, or state machine. A processor may also be implemented as a combination of computing devices, e.g., a combination of a DSP and a microprocessor, a plurality of microprocessors, one or more microprocessors in conjunction with a DSP core, or any other such configuration.

The steps of a method or algorithm described in connection with the configurations disclosed herein may be configured directly in hardware, in a software module executed by a processor, or in a combination of the two. A software module may reside in RAM memory, flash memory, ROM memory, EPROM memory, EEPROM memory, registers, hard disk, a removable disk, a CD-ROM, or any other form of storage medium known in the art. An exemplary storage medium is coupled to the processor such that the processor can read information from, and write information to, the storage medium. In the alternative, the storage medium may be integral to the processor. The processor and the storage medium may reside in an ASIC. The ASIC may reside in a user terminal. In the alternative, the processor and the storage medium may reside as discrete components in a user terminal.

The methods disclosed herein comprise one or more steps or actions for achieving the described method. The method steps and/or actions may be interchanged with one another without departing from the scope of the present invention. In other words, unless a specific order of steps or actions is required for proper operation of the embodiment, the order and/or use of specific steps and/or actions may be modified without departing from the scope of the present invention.

While specific configurations and applications of the present invention have been illustrated and described, it is to be understood that the invention is not limited to the precise configuration and components disclosed herein. Various modifications, changes, and variations which will be apparent to those skilled in the art may be made in the arrangement, operation, and details of the methods and systems of the present invention disclosed herein without departing from the spirit and scope of the invention.

What is claimed is:

1. A method for scheduling a medical service employing at least one computer system, comprising:
receiving a request for a medical service at the computer system;
scheduling the medical service for a requested date;
analyzing at least one nonpayment exposure factor to determine whether providing the medical service involves a nonpayment exposure to a medical service provider; and
rescheduling the medical service for a date after the requested date if the analysis indicates the nonpayment exposure to the medical service provider.

2. The method of claim 1, wherein the scheduled date for the medical service is maintained if the analysis does not indicate the nonpayment exposure to the medical service provider.

3. The method of claim 1, wherein rescheduling the medical service for a date after the requested date involves scheduling the medical service at least one week after the requested date.

4. The method of claim 1, further comprising determining whether the medical service is urgent and if the medical service is urgent, scheduling the medical service for the requested date.

5. The method of claim 1, wherein the request for the medical service is received from a second computer system.

6. The method of claim 1, wherein if the medical service is not urgent, providing the service even if the analysis indicates the nonpayment exposure to the medical service provider.

7. The method of claim 1, wherein the analysis of the at least one nonpayment exposure factor is performed automatically.

8. The method of claim 1, wherein the analysis of the at least one nonpayment exposure factor comprises obtaining a weighted score that indicates the nonpayment exposure and comparing this score against a threshold.

9. A computer system for scheduling a medical service, the computer system comprising:
a processor;
memory in electronic communication with the processor;
instructions stored in the memory, the instructions being executable to:
receive a request for a medical service at the computer system;
schedule the medical service for a requested date;
analyze at least one nonpayment exposure factor to determine whether providing the medical service involves a nonpayment exposure to a medical service provider; and
reschedule the medical service for a date after the requested date if the analysis indicates the nonpayment exposure to the medical service provider.

10. The computer system of claim 9, wherein the instructions executable to reschedule the medical service for a date after the requested date comprise instructions executable to schedule the medical service at least one week after the requested date.

11. The computer system of claim 9, wherein the instructions are executable to receive the request for the medical service from a second computer system.

12. The computer system of claim 9, wherein the analysis of the at least one nonpayment exposure factor is performed automatically.

13. The computer system of claim 9, wherein the instructions are further executable to determine whether the medical service is urgent and if the medical service is urgent, the instructions are executable to maintain the scheduled date for the medical service scheduled.

14. A computer-readable medium comprising instructions stored in memory and executable by a processor for scheduling a medical service, the instructions being executable to:
receive a request for a medical service at the computer system;
schedule the medical service for a requested date;
analyze at least one nonpayment exposure factor to determine whether providing the medical service involves a nonpayment exposure to a medical service provider; and
reschedule the medical service for a date after the requested date if the analysis indicates the nonpayment exposure to the medical service provider.

15. The computer-readable medium of claim 14, wherein the instructions are executable to receive the request for the medical service from a second computer system.

16. The computer-readable medium of claim 14, wherein the instructions are further executable to determine whether the medical service is urgent and if the medical service is urgent, the instructions are executable to maintain the scheduled date for the medical service.

17. The computer-readable medium of claim 14, wherein the instructions to analyze at least one nonpayment exposure factor comprises instructions executable to obtain a weighted score that indicates that nonpayment exposure and instructions executable to compare this score against a threshold.
